(19) United States
(12) Patent Application Publication
(10) Pub. No.: US 2019/0290830 A1
(43) Pub. Date: Sep. 26, 2019

(54) BIOLOGICAL COMPONENT TREATMENT CASSETTE AND BIOLOGICAL COMPONENT TREATMENT SYSTEM

(71) Applicant: TERUMO KABUSHIKI KAISHA, Tokyo (JP)

(72) Inventor: Masatsugu IGARASHI, Shizuoka (JP)

(73) Assignee: TERUMO KABUSHIKI KAISHA, Tokyo (JP)

(21) Appl. No.: 16/362,360

(22) Filed: Mar. 22, 2019

(30) Foreign Application Priority Data
Mar. 26, 2018 (JP) 2018-057619

(51) Int. Cl. A61M 1/36; B04B 3/00; B04B 5/04; B04B 11/02

(52) U.S. Cl. CPC A61M 1/3693; A61M 1/3607; B04B 3/00; A61M 2205/3331; B04B 11/02; A61M 2205/3317; A61M 2205/125; B04B 5/0442

(57) ABSTRACT

A biological component treatment cassette (blood component collection cassette (28)) is configured to be attachable to a separation device (centrifugal separation device (14)) adapted to separate a biological component from a liquid containing at least one biological component, and a flow path (42) is formed in an interior portion thereof. The biological component treatment cassette includes a sheet-shaped cassette main body (40), and a first pump action tube (58), both ends of which are connected to the flow path (42) of the cassette main body (40), together with a portion thereof protruding from an outer peripheral edge portion of the cassette main body (40), and which is made from a soft material that is pressed by a pump (80) of a separation device.

### TECHNICAL FIELD

The present invention relates to a biological component treatment cassette and a biological component treatment system.

### BACKGROUND ART

In blood donation in recent years, in addition to whole blood collection in which whole blood is collected from blood donors, component blood sampling (apheresis) has been performed in which the burden on the blood donor's body is made lighter. Component blood sampling is a blood collection method in which a blood component collection system (apheresis system) is used, whereby only specific blood components are collected from whole blood, and the remaining blood components are returned again into the donor's body. For example, in Japanese Laid-Open Patent Publication No. 2013-514863 (PCT), a blood component collection system is disclosed in which blood platelets are collected by centrifugally separating by a separation device whole blood that is extracted from a blood donor.

### SUMMARY OF INVENTION

Since the cassette of the conventional blood component collection system is a molded product made of a hard resin produced by injection molding, there is a problem in that the configuration of the cassette is complicated, and the cost to manufacture the cassette is high. Thus, it may be considered to construct a cassette from a soft material. In this case, it is preferable that the cassette is capable of being efficiently mounted in the separation device. Not only in a blood component collection system, but also in relation to other biological component treatment systems that perform treatment by allowing a liquid containing at least one biological component to flow therethrough, for example, in culture devices for various types of cultured cells, medicinal solution administration systems, or the like, similar problems are known to occur.

Thus, an object of the present invention is to provide a biological component treatment cassette and a biological component treatment system, which enable manufacturing costs to be reduced, and together therewith, are capable of efficiently performing mounting of the cassette in a separation device.

In order to achieve the aforementioned object, one aspect of the present invention is characterized by a biological component treatment cassette configured to be attachable to a separation device adapted to separate a biological component from a liquid containing at least one biological component, and having a flow path formed in an interior portion thereof, the biological component treatment cassette comprising a sheet-shaped cassette main body on which there is formed a flow path wall configured to form the flow path in an interior portion thereof, and a pump action tube, both ends of which are connected to the flow path of the cassette main body, together with a portion thereof protruding from an outer peripheral edge portion of the cassette main body, and which is made from a soft material that is pressed by a pump installed in the separation device in order to cause a fluid to flow inside the flow path.

In accordance with such a biological component treatment cassette, since at least a portion of the cassette is made of a soft material, it is possible to reduce manufacturing costs, as compared with a conventional cassette which is made from a hard resin manufactured by injection molding. Further, since the pump action tube which is pressed by the pump of the separation device is formed integrally with the cassette, setting of the pump action tube on the pump can be carried out simply by mounting the cassette at a predetermined position of the separation device. Accordingly, it is possible tb efficiently mount the cassette in the separation device. Furthermore, since the pump action tube is disposed on the outer side of the cassette main body, vibrations of the pump are unlikely to be transmitted to the pressed portion used for detecting the load, and the freedom in arrangement of the pressed portion is improved.

Another aspect of the present invention is characterized by a biological component treatment system equipped with a separation device adapted to separate a biological component from a liquid containing at least one biological component, and a biological component treatment cassette configured to be attachable to the separation device, and having a flow path formed in an interior portion thereof, wherein the biological component treatment cassette comprises a sheet-shaped cassette main body on which there is formed a flow path wall configured to form the flow path in an interior portion thereof, and a pump action tube, both ends of which are connected to the flow path of the cassette main body, together with a portion thereof protruding from an outer peripheral edge portion of the cassette main body, and which is made from a soft material, and the separation device comprises a pump configured to press on the pump action tube in order to cause a fluid to flow inside the flow path.

According to the biological component treatment cassette and the biological component treatment system of the present invention, manufacturing costs can be reduced, and together therewith, mounting of the cassette in the separation device can be performed efficiently.

### BRIEF DESCRIPTION OF DRAWINGS

FIG. 1 is a schematic diagram of a biological component treatment system (blood component collection system) according to a first embodiment of the present invention;

FIG. 2A is a first explanatory diagram of operations of the blood component collection system and FIG. 2B is a second explanatory diagram of operations of the blood component collection system;

FIG. 3A is a third explanatory diagram of operations of the blood component collection system and FIG. 3B is a fourth explanatory diagram of operations of the blood component collection system;

FIG. 4 is a fifth explanatory diagram of operations of the blood component collection system;

FIG. 5 is a schematic view of a biological component treatment system (cell concentration and cleaning system) according to a second embodiment of the present invention;

FIG. 6A is a first explanatory diagram of operations of the cell concentration and cleaning system and FIG. 6B is a second explanatory diagram of operations of the cell concentration and cleaning system;

FIG. 7A is a third explanatory diagram of operations of the cell concentration and cleaning system and FIG. 7B is a fourth explanatory diagram of operations of the cell concentration and cleaning system;

FIG. 8A is a fifth explanatory diagram of operations of the cell concentration and cleaning system and FIG. 8B is a sixth explanatory diagram of operations of the cell concentration and cleaning system;

FIG. 9A is a seventh explanatory diagram of operations of the cell concentration and cleaning system and FIG. 9B is an eighth explanatory diagram of operations of the cell concentration and cleaning system.

### DESCRIPTION OF EMBODIMENTS

Preferred embodiments of a biological component treatment cassette and a biological component treatment system according to the present invention will be presented and described in detail below with reference to the accompanying drawings.

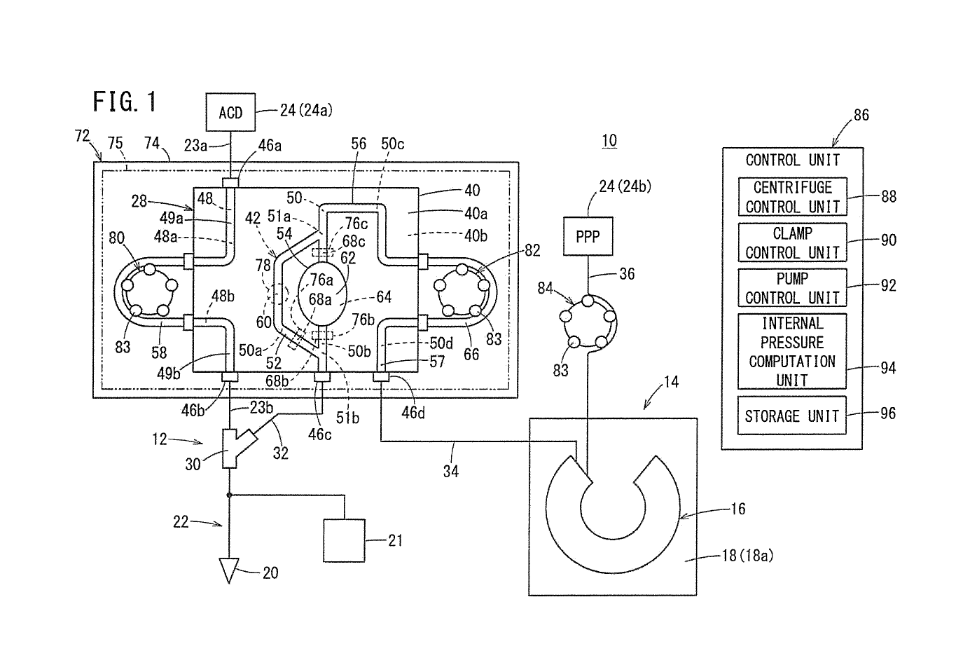

As shown in FIG. 1, a blood component collection system 10, which is one form (first embodiment) of the biological component collection system, is constituted as a blood apheresis system, in which blood (whole blood) is continuously extracted from a donor and subjected to centrifugal separation outside the body, whereby a specific blood component (in the present embodiment, plasma (platelet poor plasma: PPP)) is collected and the remaining blood components are returned to the donor. In the present embodiment, the blood is defined as “a liquid containing at least one biological component”.

The blood component collection system 10 is equipped with a blood collection circuit set 12 for enabling storage and flow of blood components therein, and a centrifugal separation device 14 (one form of a blood component separation device or a separation device) that applies a centrifugal force to the blood collection circuit set 12. The blood collection circuit set 12 includes a blood treatment unit 16 to which there is introduced whole blood that is removed from the donor, and the whole blood is centrifugally separated into a plurality of blood components. The centrifugal separation device 14 is equipped with a centrifuge unit 18 having a centrifugal rotor 18a for applying a centrifugal force to the blood treatment unit 16. The blood treatment unit 16 is capable of being mounted in the centrifuge unit 18.

The blood collection circuit set 12 is discarded every time that it is used in order to prevent contamination and ensure sanitation. The blood collection circuit set 12 comprises a blood collection and blood returning unit 22 having a blood collecting needle 20 and an initial flow blood collecting bag 21, the blood treatment unit 16, a plurality of bags 24, and a blood component collection cassette 28 (hereinafter referred to as a “cassette 28”) which is one form of the biological component treatment cassette. The plurality of bags 24 include an ACD solution bag 24a containing an ACD solution which is an anticoagulant, and a PPP bag 24b for storing the plasma (platelet poor plasma).

The blood collection and blood returning unit 22 is connected to the ACD solution bag 24a and the cassette 28 via a tube connector 30. The ACD solution bag 24a is connected to the cassette 28 via a first ACD solution transfer tube 23a.

The cassette 28 is connected to the blood collection and blood returning unit 22 via a donor side tube 32, and is also connected to the blood treatment unit 16 via a treatment unit side tube 34. Further, the cassette 28 is connected to the tube connector 30 via a second ACD solution transfer tube 23b. The blood treatment unit 16 is attached to the centrifuge unit 18 (centrifugal rotor 18a) of the centrifugal separation device 14, and is configured in the form of a container in which blood can be introduced therein, flow therethrough, and flow out therefrom. The PPP bag 24b is connected to the blood treatment unit 16 via a PPP transfer tube 36.

The cassette 28 is equipped with a cassette main body 40 having a flow path 42 formed therein, and at least one pump action tube (in the first embodiment, a first pump action tube 58 and a second pump action tube 66) connected to the cassette main body 40. The cassette main body 40 is formed in a rectangular shape as viewed in plan. The cassette main body 40 is formed of a soft material. For the soft material that constitutes the cassette main body 40, the same material is used over the entirety of the cassette main body 40. Moreover, the cassette main body 40 may be constituted from a plurality of different materials. More specifically, the cassette main body 40 includes a first sheet 40a and a second sheet 40b formed of a soft material. The first sheet 40a and the second sheet 40b are stacked in a thickness direction and are joined to each other.

As examples of the soft material that constitutes the first sheet 40a and the second sheet 40b, there may be cited vinyl chloride, polyolefin, polyurethane, and the like. As examples of a plasticizer for vinyl chloride, there may be cited diisononylcyclohexane-1,2-dicarboxylate, bis-2-ethylhexyl phthalate, and the like.

The flow path 42 is formed between the first sheet 40a and the second sheet 40b. Fusion bonding (high frequency fusion bonding, thermal fusion bonding, etc.) is used as the means for joining the first sheet 40a and the second sheet 40b. The first sheet 40a and the second sheet 40b may also be joined together by another joining means (adhesion or the like). Further, port members 46a to 46d, which are made of a hard material (for example, polypropylene, polycarbonate, or the like), are disposed on an outer peripheral edge portion of the cassette main body 40.

The first ACD solution transfer tube 23a is connected to the port member 46a. The second ACD solution transfer tube 23b is connected to the port member 46b. The port members 46a and 46b are disposed on opposite sides of the quadrangular shaped cassette main body 40. The donor side tube 32 and the treatment unit side tube 34 are connected respectively to the port members 46c and 46d. The port members 46b to 46d are provided on the same side of the cassette main body 40.

The flow path 42 that is formed in the cassette main body 40 includes a first flow path (hereinafter referred to as an “ACD solution flow path 48”) through which the ACD solution flows when the centrifugal separation device 14 is in operation, and a blood flow path 50 which is independent of the ACD solution flow path 48, and in which blood or blood components flow when the centrifugal separation device 14 is in operation. The ACD solution flow path 48 is configured substantially in the shape of a straight line (I-shape) and communicates with the port member 46a. The blood flow path 50 is configured substantially in the form of a U-shape, and communicates with the port members 46c and 46d.

The ACD solution flow path 48 includes a first ACD line 48a communicating with the first ACD solution transfer tube 23a, and a second ACD line 48b communicating with the second ACD solution transfer tube 23b. The blood flow path 50 includes a first line 50a, a second line 50b connected in parallel with respect to the first line 50a, a third line 50c connected in series with respect to the first line 50a and the second line 50b, and a fourth line 50d separated from the first line 50a, the second line 50b, and the third line 50c. The first line 50a and the second line 50b are connected via a first coupling member 51a and a second coupling member 51b.

The cassette main body 40 includes a first ACD line forming member 49a forming the first ACD line 48a, and a second ACD line forming member 49b forming the second ACD line 48b. Further, the cassette main body 40 comprises a first line forming member 52 forming the first line 50a, a second line forming member 54 forming the second line 50b, a third line forming member 56 forming the third line 50c, and a fourth line forming member 57 forming the fourth line 50d.

The first line forming member 52 includes a pressed portion 60 made of a soft material. In order to measure the internal pressure of the flow path 42 during operation of the centrifugal separation device 14 in a state (hereinafter referred to as a “cassette attached state”) in which the cassette 28 is attached to the centrifugal separation device 14, the pressed portion 60 is a site that is pressed by a later-described load detecting unit 78 which is installed in the centrifugal separation device 14. Accordingly, the pressed portion 60 is a measured portion where the load applied to the flow path wall is measured by the load detecting unit 78. The pressed portion 60 may be configured to include a magnetic sensor, which is provided in the cassette main body 40 that is formed in a sheet-like shape in a manner so as to follow the deformation of the flow path 42.

The second line forming member 54 includes a filter accommodating unit 62 which is made of a soft material. A filter member 64 in the form of a sheet mesh is disposed inside the filter accommodating unit 62 for the purpose of removing clotted blood or blood clumps contained within the blood or the blood components.

In the cassette main body 40, even if there is no positive pressure acting inside the flow path 42, the wall portions (flow path walls) that form the flow path 42 bulge in convex shapes in the thickness direction of the cassette 28 on both side surfaces of the cassette main body 40. Accordingly, the wall portions (the first ACD line forming member 49a and the second ACD line forming member 49b) that form the ACD solution flow path 48, and the wall portions (the first line forming member 52, the second line forming member 54, the third line forming member 56, and the fourth line forming member 57) that form the blood flow path 50 bulge in convex shapes in the thickness direction of the cassette 28. The flow path 42 is a flow path which is opened in its natural state. When pressed by an external force, the wall portions can be elastically deformed in directions to close the flow path 42 at the pressed locations thereof.

On the cassette 28, there are provided a plurality of clamp action members 68a to 68c on which a plurality of clamps 76a to 76c which are flow path opening/closing mechanisms provided in the centrifugal separation device 14 act. When the cassette 28 is installed in the centrifugal separation device 14, the clamp action members 68a to 68c abut against or are placed in facing relation to their corresponding clamps 76a to 76c. The clamp action member 68a is provided in the first line forming member 52. Within the second line forming member 54, the clamp action member 68b is provided at an end portion thereof on a side in close proximity to the port member 46c. Within the second line forming member 54, the clamp action member 68c is provided at an end portion thereof on a side remote from the port member 46c.

Moreover, the flow path structure formed in the cassette 28, and the number and arrangement of the bags 24 that are provided are not limited to the configurations shown and described above, but may be modified in accordance with the type of blood components to be collected, the method of use, and the like.

The first pump action tube 58 and the second pump action tube 66 communicate with the flow path 42 of the cassette main body 40, and together therewith, project from an outer peripheral edge portion of the cassette main body 40. The first pump action tube 58 and the second pump action tube 66 are made of a soft material. Internal flow paths of each of the first pump action tube 58 and the second pump action tube 66 define flow paths that are open in their natural state. When pressed by an external force, the first pump action tube 58 and the second pump action tube 66 are capable of being elastically deformed in a direction to close the internal flow paths at the pressed locations thereof. The first pump action tube 58 and the second pump action tube 66 are preferably made of a material that is harder than the soft material constituting the cassette main body 40.

The first pump action tube 58 and the second pump action tube 66 are disposed respectively on opposite sides of the cassette main body 40.

More specifically, both end portions of the first pump action tube 58 are connected to the cassette main body 40. One end portion of the first pump action tube 58 communicates with the first ACD line 48a. Another end portion of the first pump action tube 58 communicates with the second ACD line 48b. The first pump action tube 58 is curved in a U-shape.

Both end portions of the second pump action tube 66 are connected to the cassette main body 40. One end portion of the second pump action tube 66 communicates with the third line 50c of the blood flow path 50. Another end portion of the second pump action tube 66 communicates with the fourth line 50d of the blood flow path 50. The second pump action tube 66 is curved in a U-shape.

The centrifugal separation device 14 is a device that is used repeatedly during blood component collection, and is provided, for example, in a medical facility, a blood collection vehicle, or the like. The centrifugal separation device 14 is equipped with the centrifuge unit 18 (separation processing unit) having the centrifugal rotor 18a, and a cassette mounting unit 72 to which the cassette 28 of the blood collection circuit set 12 is capable of being attached.

The cassette mounting unit 72 includes an attachment base 74 having a cassette mounting groove formed therein, a lid 75 which can be opened and closed and is configured in a manner so as to cover the attachment base 74 when closed, the plurality of clamps 76a to 76c which are configured to be capable of pressing on the clamp action members 68a to 68c of the cassette 28, and the load detecting unit 78 which is capable of pressing on the pressed portion 60 of the cassette 28.

The lid 75, for example, is connected in a rotatable manner to the attachment base 74. When the lid 75 is closed with the cassette 28 being held in the cassette mounting groove of the attachment base 74, the cassette 28 is sandwiched between the attachment base 74 and the lid 75.

The plurality of clamps 76a to 76c are capable of being advanced and retracted in the thickness direction of the cassette 28 in a state in which the cassette 28 is retained in the cassette mounting groove of the attachment base 74, and are disposed corresponding to the arrangement of the plurality of clamp action members 68a to 68c provided on the cassette 28.

At a time that the clamp action members 68a to 68c are not being pressed by the clamps 76a to 76c in a state in which the cassette 28 is mounted in the cassette mounting unit 72, the flow paths inside the clamp action members 68a to 68c are opened. When the clamps 76a to 76c press on the clamp action members 68a to 68c, the flow paths in the interior of the clamp action members 68a to 68c are closed. In addition, when the clamps 76a to 76c are retracted, due to the elastic restorative force of (the clamp action members 68a to 68c of) the cassette main body 40, the clamp action members 68a to 68c are restored to their original shape, and the flow paths inside the clamp action members 68a to 68c are opened.

The centrifugal separation device 14 comprises a first pump 80 that acts on the first pump action tube 58 provided on the cassette 28, a second pump 82 that acts on the second pump action tube 66 provided on the cassette 28, and a third pump 84 that acts on the PPP transfer tube 36.

The first pump 80 is a pump that transfers the ACD solution from the ACD solution bag 24a to the blood treatment unit 16 via the cassette 28. The second and third pumps 82 and 84 are pumps that transfer blood from the side of the donor to the blood treatment unit 16, together with transferring blood components from the blood treatment unit 16 to the side of the donor.

The first pump 80 and the second pump 82 are peristaltic pumps, which press respectively on the first pump action tube 58 and the second pump action tube 66 to thereby cause peristaltic movement therein. More specifically, the first pump 80 and the second pump 82 are configured in a manner so as to press respectively on the first pump action tube 58 and the second pump action tube 66 of the cassette 28 that is held in the cassette mounting unit 72, whereby the liquid (the ACD solution, the blood or the blood components) inside the cassette 28 is made to flow. The third pump 84 is configured in a manner so as to press on the PPP transfer tube 36, whereby the liquid in the PPP transfer tube 36 is made to flow.

In the first embodiment, the first pump 80, the second pump 82, and the third pump 84 are constituted by peristaltic pumps (so-called roller pumps). Such peristaltic pumps are pumps that include a plurality of pressing rollers 83, and perform liquid feeding by the plurality of pressing rollers 83 moving on the circumference of the tubes while pressing on the tubes.

Accompanying the attachment of the cassette 28 to the cassette mounting unit 72, the first pump action tube 58 is disposed in a manner so that the first pump 80 is located on an inner side of the curved shape of the first pump action tube 58, and the second pump action tube 66 is disposed in a manner so that the second pump 82 is located on an inner side of the curved shape of the second pump action tube 66. The third pump 84 may also be constituted by a peristaltic pump.

The centrifugal separation device 14 further includes a control unit 86. The control unit 86 includes a centrifuge control unit 88 for controlling the centrifuge unit 18, a clamp control unit 90 for controlling the clamps 76a to 76c, a pump control unit 92 for controlling the pumps 80, 82, and 84, an internal pressure computation unit 94 that acquires (calculates) a circuit internal pressure of the blood collection circuit set 12, and a storage unit 96 in which predetermined information is stored.

Next, operations of the blood component collection system 10 according to the present embodiment, which is configured in the manner described above, will be described.

As a preparation (set-up) for collecting blood components from the donor using the blood component collection system 10 shown in FIG. 1, the blood collection circuit set 12 is attached to the centrifugal separation device 14. More specifically, the cassette 28 is mounted in the cassette mounting unit 72, and the blood treatment unit 16 is attached to the centrifugal rotor 18a. On the other hand, the blood collecting needle 20 pierces and is inserted into the donor.

When the cassette 28 is mounted in the cassette mounting unit 72, at first, the cassette 28 is mounted in the cassette mounting groove provided in the attachment base 74. In addition, by closing the lid 75, the cassette 28 is placed in a state of being held between the lid 75 and the attachment base 74. As a result, the pressed portion 60 of the cassette 28 is pressed by the load detecting unit 78 and is placed in a state of being slightly elastically deformed. Further, the plurality of clamp action members 68a to 68c of the cassette 28 are placed in facing relation with respect to the plurality of clamps 76a to 76c. Furthermore, the first pump action tube 58 and the second pump action tube 66 are attached respectively to the first pump 80 and the second pump 82.

When a command is issued by operation of a user with respect to the centrifugal separation device 14 in order to initiate operations, in the centrifugal separation device 14, under the action of the first pump 80, priming with the ACD solution is carried out.

Next, by rotating the centrifugal rotor 18a, the centrifugal separation device 14 applies a centrifugal force to the blood treatment unit 16 that is attached to the centrifugal rotor 18a, and together therewith, by operation of the second pump 82, blood (whole blood) from the donor is extracted and introduced into the blood treatment unit 16 (blood collection operation). By the centrifugal force that accompanies rotation of the centrifugal rotor 18a, the blood introduced into the blood treatment unit 16 is separated into red blood cells (concentrated red blood cells), a buffy coat, and plasma (platelet poor plasma).

The plasma that is separated in the blood treatment unit 16 is introduced into the PPP bag 24b via the PPP transfer tube 36. After completion of the centrifugal separation process, the remaining blood components (the red blood cells and the buffy coat) are returned to the donor (returning operation). At this time, since foreign substances such as blood clumps and the like contained within the remaining blood components are trapped by the filter member 64 provided in the second line 50b of the cassette 28, any risk of such foreign matter being returned to the donor can be reduced. The collection operation and the returning operation described above are repeated a plurality of times.

During the centrifugal separation process, the centrifugal separation device 14 measures the circuit internal pressure (negative pressure and positive pressure) on the basis of the load detected by the load detecting unit 78. The circuit internal pressure is calculated by the internal pressure computation unit 94 of the control unit 86. The storage unit 96 stores a calibration curve indicating a relationship between the detection value (load) of the load detecting unit 78 and the pressure value. The internal pressure computation unit 94 calculates the circuit internal pressure with reference to the calibration curve. The calculated (measured) circuit internal pressure, for example, ranges from −300 to 500 mmHg. In order to more accurately measure the circuit internal pressure, for example, in addition to the load detecting unit 78, another load detecting unit 78 may also be provided in the centrifugal separation device 14, and the aforementioned calibration curve may be corrected. The reference data used when calculating the circuit internal pressure using the load detected by the load detecting unit 78 is not limited to the calibration curve, but may be a table that is prepared beforehand.

During operation of the blood component collection system 10, specifically, the centrifugal separation device 14 is operated in the following manner.

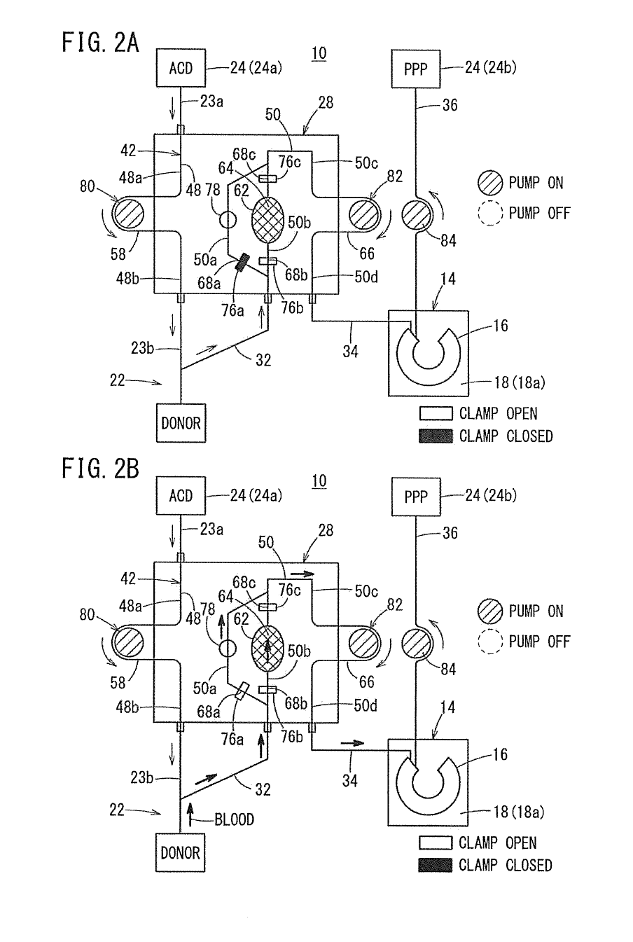

As shown in FIG. 2A, when priming with the ACD solution is carried out, the clamp 76a is closed, and the clamps 76b and 76c are opened. Additionally, in this state, the pumps 80, 82, and 84 are operated. By action of the first pump 80, the ACD solution flows from the ACD solution bag 24a into the cassette 28 via the first ACD solution transfer tube 23a. In this case, the first pump 80 acts (presses) on the first pump action tube 58. In addition, the ACD solution flows to the donor side tube 32 via the second ACD solution transfer tube 23b. At a stage at which it is detected by a non-illustrated line sensor outside of the cassette 28 that the ACD solution has arrived in the immediate vicinity of the port member 46c, priming by the ACD solution is terminated.

Next, when blood collection is started for the first time, initially, as shown in FIG. 2B, all of the clamps 76a to 76c are opened. Additionally, in this state, the blood from the donor is introduced into the blood flow path 50 of the cassette 28, and all of the air inside the circuit of the cassette 28 is pushed out by the blood into the blood treatment unit 16 (cassette interior priming step). In this case, due to the action of the second pump 82, the blood flows from the donor into the cassette 28 via the donor side tube 32, and together therewith, flows into the treatment unit side tube 34 via the blood flow path 50 inside the cassette 28. The second pump 82 acts (presses) on the second pump action tube 66.

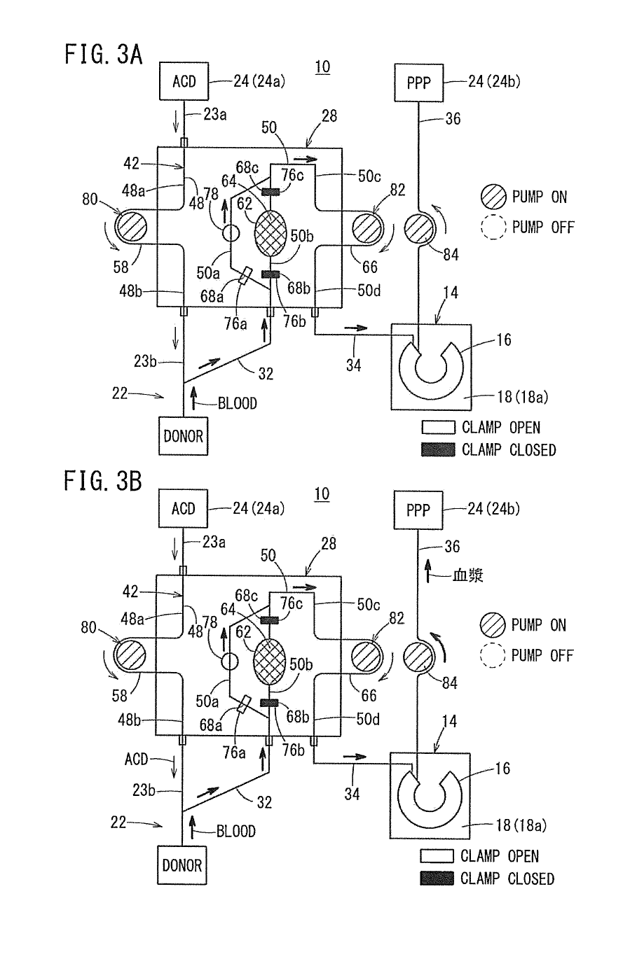

Next, as shown in FIG. 3A, by closing the clamps 76b and 76c, the second line 50b is closed. Consequently, a negative pressure is prevented from acting on the filter accommodating unit 62 and blocking the filter accommodating unit 62. The blood flows through the first line 50a and the third line 50c, and is introduced into the blood treatment unit 16. Accompanying introduction of the blood into the blood treatment unit 16, the blood treatment unit 16 initiates a centrifugal operation, and the blood is subjected to centrifugal separation in the blood treatment unit 16. As shown in FIG. 3B, by action of the third pump 84, the plasma that is separated from the blood in the blood treatment unit 16 is introduced into the PPP bag 24b via the PPP transfer tube 36.

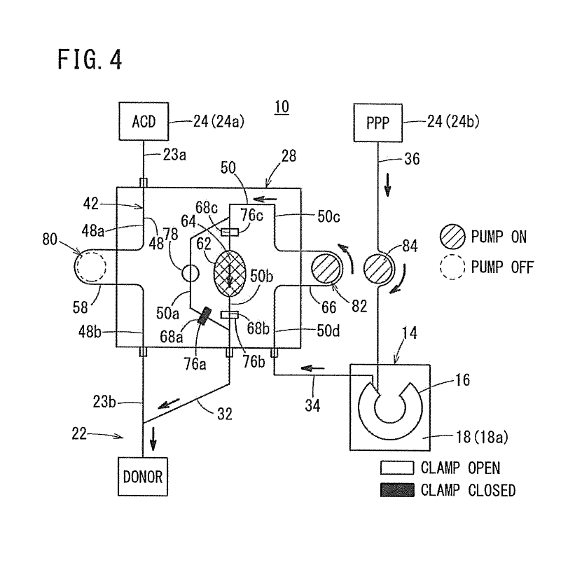

When a predetermined amount of plasma has been stored in the PPP bag 24b, the blood components (in the present embodiment, blood cell components) are returned to the donor (blood returning step). In the blood returning step, as shown in FIG. 4, the clamp 76a is closed, and the clamps 76b and 76c are opened, whereby the first line 50a is closed, whereas the second line 50b is opened. Additionally, in this state, a part of the collected plasma flows into the blood treatment unit 16 under the action of the second and third pumps 82 and 84, and the blood components (blood cell components) are pushed out from the blood treatment unit 16 to the side of the cassette 28 (the blood flow path 50).

The blood components pushed out from the blood treatment unit 16 are returned to the donor via the cassette 28. In this case, the blood cell components pass through the second line 50b in which the filter member 64 is arranged. When the blood components pass through the filter member 64, clotted blood contained within the blood components is trapped by the filter member 64. Since the first line 50a is closed, clotted blood cannot be returned to the donor via the first line 50a. The blood collection step (see FIGS. 3A and 3B) and the blood returning step (see FIG. 4) are repeated a plurality of times, and ultimately, the blood is returned and the treatment as a whole is brought to an end.

In this case, the cassette 28 and the blood component collection system 10 according to the first embodiment exhibit the following effects.

In accordance with the cassette 28, since at least a portion thereof is made of a soft material, it is possible to reduce manufacturing costs, as compared with a conventional cassette which is made from a hard resin manufactured by injection molding.

Further, since the first pump action tube 58 and the second pump action tube 66 which are pressed by the first pump 80 and the second pump 82 of the centrifugal separation device 14 are formed integrally with the cassette 28, setting of the first pump action tube 58 and the second pump action tube 66 on the first pump 80 and the second pump 82 can be carried out simply by mounting the cassette 28 at a predetermined position of the centrifugal separation device 14. Accordingly, it is possible to efficiently mount the cassette 28 in the centrifugal separation device 14.

Furthermore, since the first pump action tube 58 and the second pump action tube 66 are disposed on the outer side of the cassette main body 40, vibrations of the first pump 80 and the second pump 82 are unlikely to be transmitted to the pressed portion 60 used for detecting the load, and the freedom in arrangement of the pressed portion 60 is improved.

Only one pump action tube may be provided on the cassette 28.

In the cassette 28, the flow path 42 is disposed inside the sheet-shaped cassette main body 40 which is made of a soft material. In accordance with such a configuration, compared to a conventional cassette made of a hard resin and manufactured by injection molding, the cassette can be manufactured at a low cost. Accordingly, with a simple and economical configuration, it is possible to measure the circuit internal pressure of the cassette 28.

A portion for trapping clotted blood (the filter member 64) is provided inside the cassette main body 40. Consequently, the number of operations performed by the operator (a step of attaching the filter member 64) is reduced, and usability is enhanced.

In the above-described cassette 28, the flow path 42 is formed between the first sheet 40a and the second sheet 40b which are formed of a soft material, however, the structure that forms the flow path 42 is not necessarily limited to such a configuration. For example, within the cassette main body 40, the members that form the flow path 42 may be a plurality of tubes, and a plate shaped cassette base portion may be provided that supports the plurality of tubes. In this case, in the plurality of tubes, there are provided the pressed portion 60, the clamp action members 68a to 68c, and the filter accommodating unit 62. The cassette base portion is formed so that the pressed portion 60 is exposed, in a manner so that the load detecting unit 78 can press on the pressed portion 60. Further, the cassette base portion is formed with the clamp action members 68a to 68c being exposed, in a manner so that the clamps 76a to 76c can press on the clamp action members 68a to 68c.

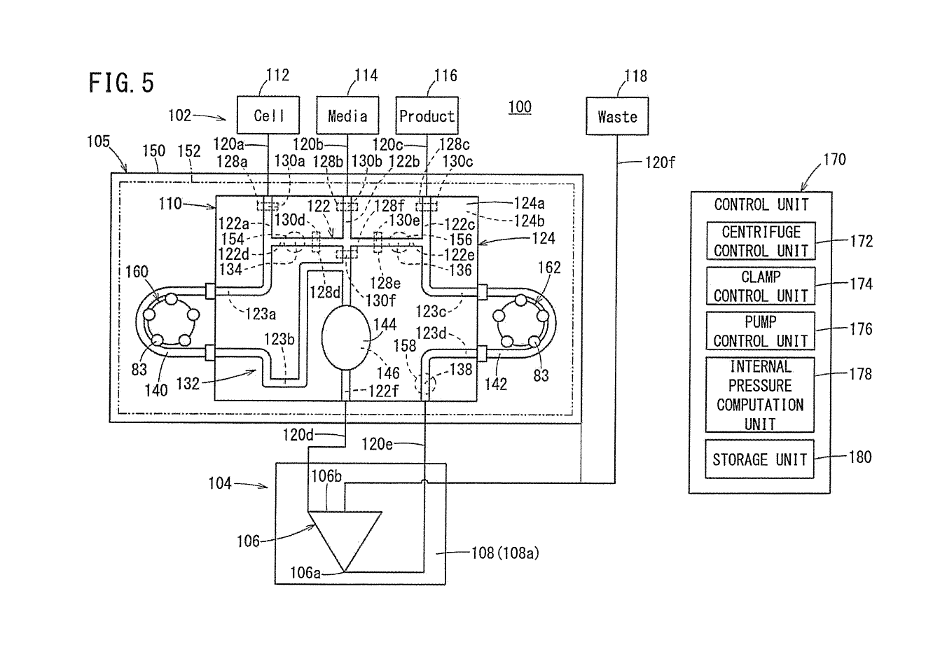

In FIG. 5, a cell concentration and cleaning system 100, which is another form (second embodiment) of the biological component treatment system, is configured as a system for concentrating and cleaning cells contained within a cell solution, and collecting the concentrated cells in a predetermined container (bag). In the second embodiment, the cell solution is “a liquid containing at least one biological component”.

The cell concentration and cleaning system 100 is equipped with a cell treatment kit 102, and a centrifuge device 104 (one form of a blood component separation device or a separation device). The cell treatment kit 102 includes a treatment chamber 106 in which the cell solution and a preservation solution are introduced, and which concentrates and cleans the cells. The centrifuge device 104 is equipped with a centrifuge unit 108 having a centrifugal rotor 108a for applying a centrifugal force to the treatment chamber 106. The treatment chamber 106 is capable of being mounted in the centrifuge unit 108.

The cell treatment kit 102 is equipped with a cell treatment cassette 110 (hereinafter simply referred to as a “cassette 110”) which is another form (second embodiment) of the biological component treatment cassette, a cell bag 112 in which the cell solution is stored, a preservation solution bag 114 in which the preservation solution is stored, a collection bag 116 for accommodating (storing) the concentrated and cleaned cells, a waste liquid bag 118 for accommodating the preservation solution after usage thereof, and the above-described treatment chamber 106.

The cell bag 112 is connected to the cassette 110 via a tube 120a. The preservation solution bag 114 is connected to the cassette 110 via a tube 120b. The collection bag 116 is connected to the cassette 110 via a tube 120c. The waste liquid bag 118 is connected to the treatment chamber 106 via a tube 120f. The treatment chamber 106 is connected to the cassette 110 via tubes 120d and 120e.

The cassette 110 is equipped with a cassette main body 124 having a flow path 122 formed therein, and at least one pump action tube (in the second embodiment, a first pump action tube 140 and a second pump action tube 142) connected to the cassette main body 124. The cassette main body 124 is formed in a rectangular shape as viewed in plan. The cassette main body 124 includes a first sheet 124a and a second sheet 124b formed of a soft material. The first sheet 124a and the second sheet 124b are stacked in a thickness direction and are joined to each other. The flow path 122 is formed between the first sheet 124a and the second sheet 124b. In the cassette main body 124, even if there is no positive pressure acting within the flow path 122, the wall portions that form the flow path 122 bulge in convex shapes in the thickness direction of the cassette 110 on both side surfaces of the cassette main body 124.

The flow path 122 includes a first line 122a communicating with the tube 120a, a second line 122b communicating with the tube 120b, a third line 122c communicating with the tube 120c, a first connecting line 122d connecting the first line 122a and the second line 122b, a second connecting line 122e connecting the second line 122b and the third line 122c, and a filter line 122f in which a filter member 146 is disposed.

The flow path 122 further includes a pump relay line 123a which is connected to the first line 122a and the first connecting line 122d, a pump relay line 123b which is connected to the filter line 122f, a pump relay line 123c which is connected to the third line 122c and the second connecting line 122e, and a pump relay line 123d that is separated from the other lines provided in the cassette main body 124.

On the cassette main body 124, there are provided a plurality of clamp action members 130a to 130f on which a plurality of clamps 128a to 128f which are flow path opening/closing mechanisms provided in the centrifuge device 104 act. In the cassette main body 124, the clamp action member 130a is provided at a location forming the first line 122a, within a convex wall portion (hereinafter referred to as a “flow path forming member 132”) that forms the flow path 122. The clamp action member 130b is provided at a location forming the second line 122b within the flow path forming member 132. The clamp action member 130c is provided at a location forming the third line 122c within the flow path forming member 132.

The clamp action member 130d is provided at a location forming the first connecting line 122d within the flow path forming member 132. The clamp action member 130e is provided at a location forming the second connecting line 122e within the flow path forming member 132. The clamp action member 130f is provided at a location forming the filter line 122f within the flow path forming member 132 (more specifically, at a location between a point of connection between the second line 122b, the first connecting line 122d, and the second connecting line 122e, and a point of connection between the filter line 122f and the pump relay line 123b).

A first pressed portion 134 made of a soft material is provided at a location forming the first connecting line 122d within the flow path forming member 132. A second pressed portion 136 made of a soft material is provided at a location forming the second connecting line 122e within the flow path forming member 132. A third pressed portion 138 made of a soft material is provided at a location forming the pump relay line 123d within the flow path forming member 132. The first pressed portion 134, the second pressed portion 136, and the third pressed portion 138 are configured in the same manner as the pressed portion 60 of the first embodiment.

A reservoir 144 made of a soft material is provided at a location forming the filter line 122f within the flow path forming member 132. The filter member 146 is accommodated inside the reservoir 144.

As the cells contained within the cell solution that is accommodated in the cell bag 112, there may be cited, for example, mesenchymal stem cells, hematopoietic stem cells, and the like. As the preservation solution that is accommodated in the preservation solution bag 114, there may be cited, for example, a DMSO-containing cell cryopreservation solution or the like.

The first pump action tube 140 and the second pump action tube 142 communicate with the flow path 122 of the cassette main body 124, and together therewith, project from an outer peripheral edge portion of the cassette main body 124. The first pump action tube 140 and the second pump action tube 142 are configured in the same manner as the first pump action tube 58 and the second pump action tube 66 in the first embodiment.

The first pump action tube 140 and the second pump action tube 142 are disposed respectively on opposite sides of the cassette main body 40.

More specifically, both end portions of the first pump action tube 140 are connected to the cassette main body 124. One end portion of the first pump action tube 140 communicates with the pump relay line 123a. Another end portion of the first pump action tube 140 communicates with the pump relay line 123b.

Both end portions of the second pump action tube 142 are connected to the cassette main body 124. One end portion of the second pump action tube 142 communicates with the pump relay line 123c. Another end portion of the second pump action tube 142 communicates with the pump relay line 123d.

The treatment chamber 106 is a container having a liquid storage chamber (treatment chamber) of a predetermined capacity in the interior thereof, and is attached to the centrifuge unit 108 of the centrifuge device 104, and subjected to a centrifugal force by the centrifuge unit 108. The treatment chamber 106 has a substantially conical shape, one end side of which is of a small diameter and the other end side of which is of a large diameter, and a location corresponding to a tip end of the conical shape constitutes a bottom part 106a forming an outer side in the centrifugal direction.

The bottom part 106a of the treatment chamber 106 is connected to the pump relay line 123d of the cassette 110 via the tube 120e. A location (hereinafter referred to as an “upper part 106b”) on a side (a side in the counter-centrifugal direction) opposite to the bottom part 106a of the treatment chamber 106 is connected to the filter line 122f of the cassette 110 via the tube 120d, together with being connected to the waste liquid bag 118 via the tube 120f.

The centrifuge device 104 is equipped with a cassette mounting unit 105 configured to enable attachment of the cassette 110 thereto, and the plurality of clamps 128a to 128f which are configured to be capable of pressing respectively on the plurality of clamp action members 130a to 130f of the cassette 110. The centrifuge device 104 is further equipped with first to third load detecting units 154, 156, and 158 which are capable of pressing respectively on the first to third pressed portions 134, 136, and 138 of the cassette 110, a first pump 160 that acts on the first pump action tube 140 provided on the cassette 110, and a second pump 162 that acts on the second pump action tube 142 provided on the cassette 110.

The cassette mounting unit 105 includes an attachment base 150 having a cassette mounting groove formed therein, and a lid 152 which can be opened and closed and is configured in a manner so as to cover the attachment base 150 when closed. The lid 152, for example, is connected in a rotatable manner to the attachment base 150. When the lid 152 is closed with the cassette 110 being held in the cassette mounting groove of the attachment base 150, the cassette 110 is sandwiched between the attachment base 150 and the lid 152.

The plurality of clamps 128a to 128f are configured in the same manner as the plurality of clamps 76a to 76c in the first embodiment. The first to third load detecting units 154, 156, and 158 are configured in the same manner as the load detecting unit 78 in the first embodiment. The first pump 160 and the second pump 162 are configured in the same manner as the first pump 80 and the second pump 82 in the first embodiment.

The centrifuge device 104 further comprises a control unit 170. The control unit 170 includes a centrifuge control unit 172 for controlling the centrifuge unit 108, a clamp control unit 174 for controlling the plurality of clamps 128a to 128f, a pump control unit 176 for controlling the first and second pumps 160 and 162, an internal pressure computation unit 178 that acquires (calculates) a circuit internal pressure of the cell treatment kit 102, and a storage unit 180 in which predetermined information is stored.

On the basis of the loads detected by the first to third load detecting units 154, 156, and 158, the centrifuge device 104 in operation measures the circuit internal pressure (negative pressure and positive pressure) at each position. The circuit internal pressure is calculated by the internal pressure computation unit 178. The storage unit 180 stores a calibration curve indicating a relationship between the detection values (loads) of the first to third load detecting units 154, 156, and 158 and the pressure value. The internal pressure computation unit 178 calculates the circuit internal pressure with reference to the calibration curve.

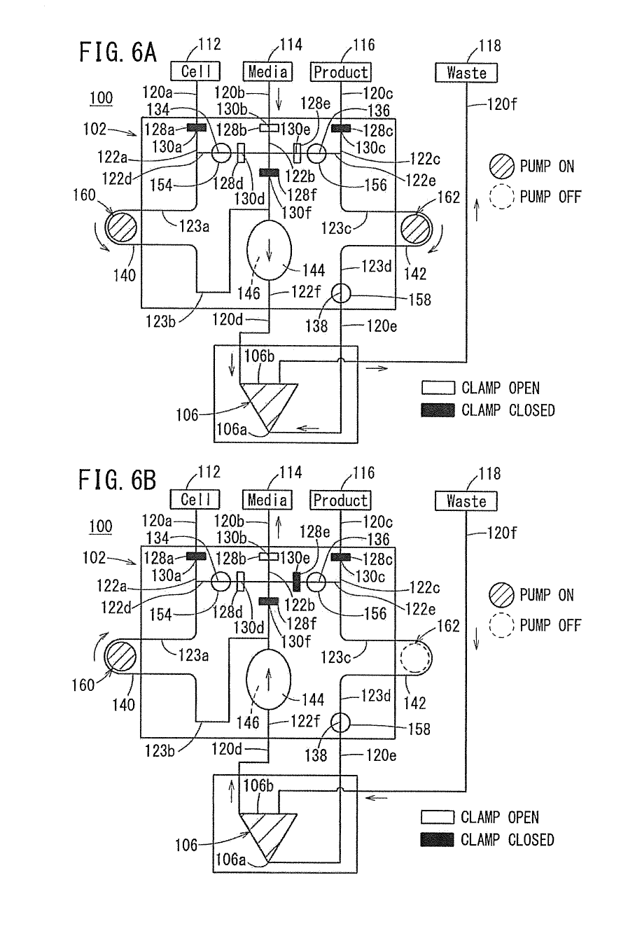

Next, with reference to FIGS. 6A to 9B, operations of the cell concentration and cleaning system 100, which is configured in the foregoing manner, will be described.

First, as shown in FIG. 6A, the cell concentration and cleaning system 100 performs a priming step of pushing the air inside the circuit of the cell treatment kit 102 with the preservation solution, and filling the interior of the circuit with the preservation solution. More specifically, a state is brought about in which the clamps 128b, 128d, and 128e are opened, and the clamps 128a, 128c, and 128f are closed. Additionally, in this state, when the first pump 160 and the second pump 162 are driven in a forward direction, the preservation solution flows through the second line 122b, the first connecting line 122d, the first pump action tube 140, and the filter line 122f, and flows into the treatment chamber 106 via the tube 120d. Further, the preservation solution flows through the second line 122b, the second connecting line 122e, and the second pump action tube 142, and flows into the treatment chamber 106 via the tube 120e. The preservation solution flows out from the treatment chamber 106 to the tube 120f, and flows into the waste liquid bag 118 via the tube 120f.

Next, as shown in FIG. 6B, in order to remove the air inside the reservoir 144, the cell concentration and cleaning system 100 performs a reservoir priming step in which the preservation solution flows into the reservoir 144 in a reverse direction to that of the priming step of FIG. 6A. More specifically, a state is brought about in which the clamps 128b and 128d are opened, and the clamps 128a, 128c, 128e, and 128f are closed. Additionally, in this state, when the first pump 160 is driven in the reverse direction, the preservation solution inside the reservoir 144 flows from the side of the tube 120d toward the side of the pump relay line 123b. It should be noted that the reservoir priming step may be omitted.

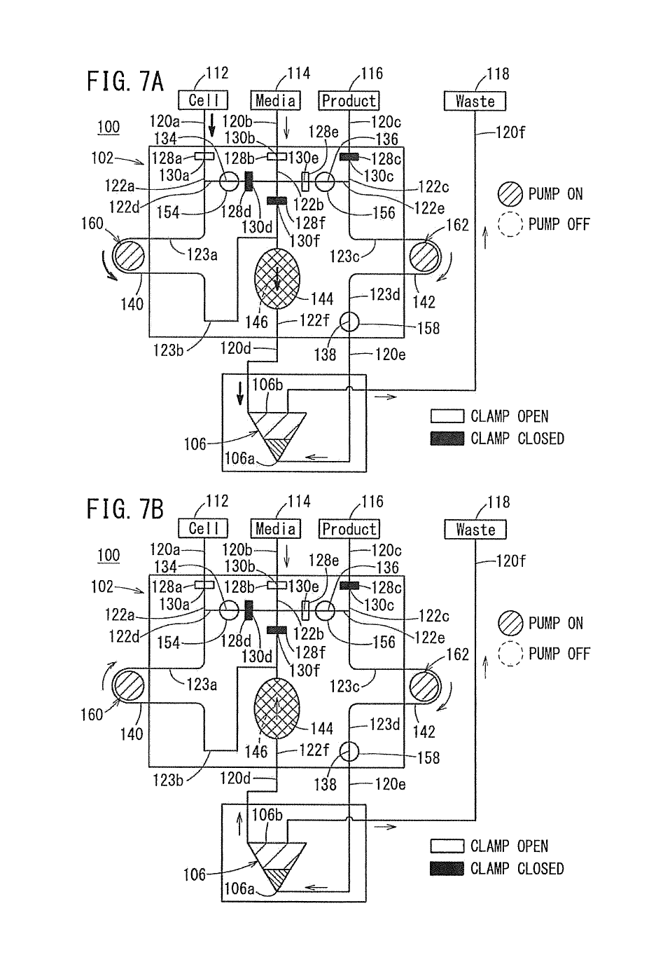

Next, as shown in FIG. 7A, the cell concentration and cleaning system 100 performs a first cell introduction step of concentrating and cleaning the cells in the treatment chamber 106. More specifically, a state is brought about in which the clamps 128a, 128b, and 128e are opened, and the clamps 128c, 128d, and 128f are closed. Additionally, in this state, the first pump 160 and the second pump 162 are driven in the forward direction. Consequently, the cell solution flows out from the cell bag 112, flows through the first line 122a, the first pump action tube 140, and the filter line 122f, and flows into the treatment chamber 106 from the upper part 106b via the tube 120d. On the other hand, the preservation solution flows out from the preservation solution bag 114, flows through the second line 122b, the second connecting line 122e, and the second pump action tube 142, and flows into the treatment chamber 106 from the bottom part 106a via the tube 120e.

By rotation of the centrifugal rotor 108a of the centrifuge unit 108, a centrifugal force acts on the cell solution that was introduced into the treatment chamber 106. Consequently, the cells move to the bottom part 106a of the treatment chamber 106 and are accumulated in the bottom part 106a. During the centrifugation process, the preservation solution is introduced into the treatment chamber 106 from the bottom part 106a of the treatment chamber 106, and a flow continues to be created from the bottom part 106a to the upper part 106b. Consequently, the cells are prevented from being excessively crushed by the centrifugal force.

Next, as shown in FIG. 7B, the cell concentration and cleaning system 100 performs a rinsing step in which the preservation solution flows into the cell bag 112 in order to collect all of the cells in the cell bag 112. More specifically, a state is brought about in which the clamps 128a, 128b, and 128e are opened, and the clamps 128c, 128d, and 128f are closed. Additionally, in this state, the first pump 160 is driven in the reverse direction, and the second pump 162 is driven in the forward direction. Consequently, the preservation solution flows out from the treatment chamber 106, flows through the filter line 122f, the first pump action tube 140, and the first line 122a of the cassette 110, and is introduced into the cell bag 112.

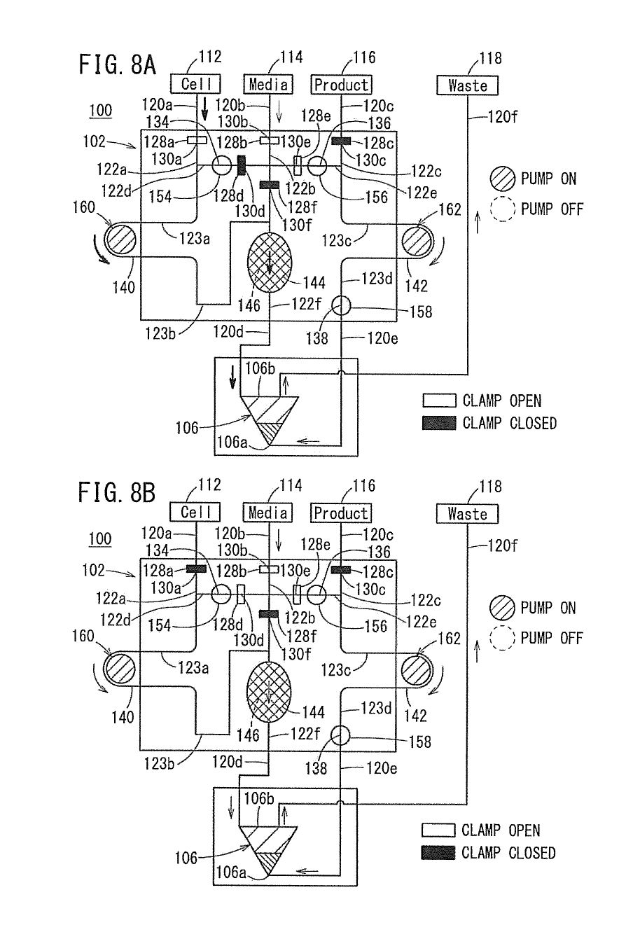

Next, as shown in FIG. 8A, the cell concentration and cleaning system 100 performs a second cell introduction step in which the cell solution (the cell solution to which the preservation solution introduced in the rinsing step has been added) is once again introduced into the treatment chamber 106.

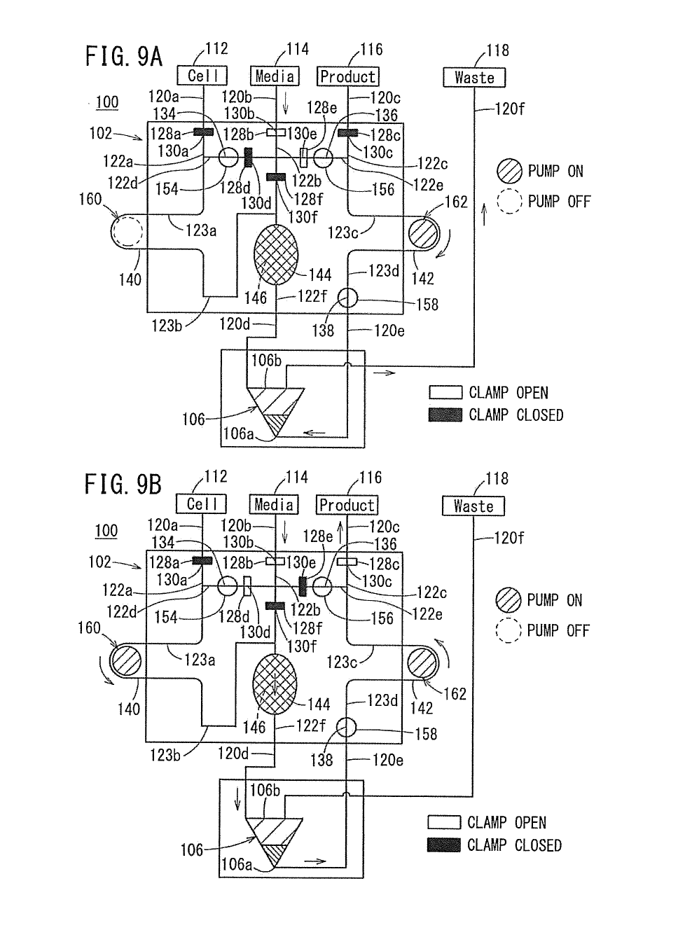

Next, as shown in FIG. 8B, the cell concentration and cleaning system 100 performs a first cleaning step in which all of the cell solution inside the flow path of the cassette 110 is pushed out by the preservation solution. More specifically, the clamps 128b, 128d, and 128e are opened and the clamps 128a, 128c, and 128f are closed, and the first pump 160 and the second pump 162 are driven in the forward direction. Following the first cleaning step, as shown in FIG. 9A, the cell concentration and cleaning system 100 continues to introduce the preservation solution from the bottom part 106a of the treatment chamber 106, and performs a second cleaning step of carrying out concentration and cleaning of the cells inside the treatment chamber 106. More specifically, a state is brought about in which the clamps 128b and 128e are opened and the clamps 128a, 128c, 128d, and 128f are closed, and the second pump 162 is driven in the forward direction.

Finally, as shown in FIG. 9B, the cell concentration and cleaning system 100 performs a collection step of collecting the cells in the collection bag 116. More specifically, a state is brought about in which the clamps 128b, 128c, and 128d are opened and the clamps 128a, 128e, and 128f are closed, the first pump 160 is driven in the forward direction, and the second pump 162 is driven in the reverse direction. Consequently, the cells flow out from the bottom part 106a of the treatment chamber 106, and are introduced into the collection bag 116 via the cassette 110 (the second pump action tube 142 and the third line 122c).

With the cassette 110 and the cell concentration and cleaning system 100 according to the second embodiment, the same advantageous effects are obtained as those of the cassette 28 and the blood component collection system 10 according to the first embodiment. More specifically, in accordance with the cassette 110 and the cell concentration and cleaning system 100, it is possible to reduce manufacturing costs, as compared with a conventional cassette which is made from a hard resin manufactured by injection molding, it is possible to efficiently perform mounting of the cassette 110 in the centrifuge device 104, and while ensuring a desired flow rate, it is possible to prevent blockage of the flow path due to negative pressure.

The scope of application of the present invention is not limited to a blood component collection system or a cell concentration and cleaning system, but may be applied to various systems through which a liquid is made to flow through a flow path, for example, a whole blood donation system, or a culture apparatus for various types of cells which are collected or cultured from patients or donors, or alternatively, a medicinal solution administration system, or the like.

The present invention is not limited to the above-described embodiments, and various modifications may be adopted within a range that does not depart from the essence and gist of the present invention.

1. A biological component treatment cassette configured to be attachable to a separation device adapted to separate a biological component from a liquid containing at least one biological component, and having a flow path formed in an interior portion thereof, the biological component treatment cassette comprising:
a cassette main body formed from a flexible upper sheet and a flexible lower sheet between which there is formed a flow path wall configured to form the flow path in an interior portion thereof, said cassette main body having a flexible peripheral edge portion; and
a pump action tube, both ends of which are connected to the flow path of the cassette main body, together with a portion thereof protruding from said outer peripheral edge portion of the cassette main body, and which is made from a soft material that is pressed by a pump installed in the separation device in order to cause a fluid to flow inside the flow path.

2. The biological component treatment cassette according to claim 1, wherein a measured portion is formed on the flow path wall.

3. The biological component treatment cassette according to claim 2, wherein the measured portion is configured in a manner so as to be pressed by the load detecting unit.

4. The biological component treatment cassette according to claim 2, wherein the measured portion comprises a magnetic sensor.

5. The biological component treatment cassette according to claim 4, wherein a plurality of the pump action tubes are provided.

6. The biological component treatment cassette according to claim 5, wherein the plurality of the pump action tubes are provided respectively on opposite sides of the cassette main body.

7. The biological component treatment cassette according to any one of claims 1 to 6, wherein the pump action tube is curved in a U-shape.

8. The biological component treatment cassette according to any one of claims 1 to 7, wherein the cassette main body is made of a soft material.

9. The biological component treatment cassette according to claim 6, wherein the pump is a peristaltic pump configured to press the pump action tube to thereby cause peristaltic movement therein.

10. The biological component treatment cassette according to claim 1, wherein the liquid containing at least one biological component is blood or a blood component.

11. The biological component treatment cassette according to claim 1, wherein the liquid containing at least one biological component is a cell solution.

12. A biological component treatment system equipped with
a separation device adapted to separate a biological component from a liquid containing at least one biological component, and
a biological component treatment cassette configured to be attachable to the separation device, and having a flow path formed in an interior portion thereof;
wherein the biological component treatment cassette comprises:
a cassette main body formed from a flexible upper sheet and a flexible lower sheet between which there is formed a flow path wall configured to form the flow path in an interior portion thereof, said cassette main body having a flexible peripheral edge; and
a pump action tube, both ends of which are connected to the flow path of the cassette main body, together with a portion thereof protruding from an outer peripheral edge portion of the cassette main body, and which is made from a soft material; and
the separation device comprises a pump configured to press on the pump action tube in order to cause a fluid to flow inside the flow path.

13. The biological component treatment cassette according to claim 2 further comprising a filter accommodating unit comprised of a soft material.

14. The biological component treatment cassette according to claim 13 wherein the filter accommodating unit comprises a filter member formed of a mesh disposed inside the filter accommodating unit.

15. The biological component treatment cassette according to claim 14 wherein said flow path comprises a plurality of fluidly connected flow path segments and said measured portion and said filter accommodating unit are in parallel flow path segments.
